(19) United States
(12) Patent Application Publication
(10) Pub. No.: US 2004/0058996 A1
(43) Pub. Date: Mar. 25, 2004

(54) Usage of vinegar acid for the treatment of dermomycosis

(76) Inventor: Naum Ruvinskiy, Los Angeles, CA (US)

Correspondence Address: Naum Ruvinskiy, 1232 N. Orange Grove Ave., Apt.# 1, Los Angeles, CA, 90046, US

(21) Appl. No.: 10/208,713

(22) Filed: Sep. 23, 2002

(51) Int. Cl. A61K 31/19

(52) U.S. Cl. 514/557

(57) ABSTRACT

My invention is the usage of vinegar acid for the treatment of dermomycosis by applying a 10% solution of vinegar acid 1-3 times daily to the effected areas. This is a new and low cost treatment of dermomycosis that is readily available and does not require repeated doctor visits.

There are many known medicines for the treatment of dermomycosis. However, they are ineffective and require numerous visits to the doctor. I propose to use vinegar acid to treat dermomycosis. The usage of vinegar acid to treat dermomycosis is highly effective, readily available and does not require repeated doctor visits. And the solution of vinegar acid is approved to be used by the FDA.

To treat dermomycosis one needs to apply 10% solution of vinegar acid to the effected areas 1-3 times daily. A more concentrated solution should be applied with caution. Also, I recommend bathing the effected area in warm water for 10-15 minutes to soften the skin and after applying the vinegar acid to scrape the skin with the knife. Vinegar acid helps to get rid of dermomycosis by killing many types of skin fungus. The results of the procedure can be seen as early as after the first application.

1. What I claim as my invention is “The usage of vinegar acid for the treatment of dermomycosis”. My invention is in the field of medicine.
